(19) United States
(12) Patent Application Publication
(10) Pub. No.: US 2012/0059053 A1
(43) Pub. Date: Mar. 8, 2012

(54) COMPOUNDS OF 2,3-DIHYDRO-BENZOFURAN

(75) Inventors: José L. Falcó, Barcelona (ES); Albert Palomer, Barcelona (ES); Antonio Guglietta, Molins de Rei (ES)

(73) Assignee: FERRER INTERNATIONAL, S.A., Barcelona (ES)

(21) Appl. No.: 12/739,600

(22) PCT Filed: Oct. 23, 2008
(86) PCT No.: PCT/EP08/64393

§ 371 Date: Nov. 21, 2011

(30) Foreign Application Priority Data
Oct. 25, 2007 (ES) P200702799

(51) Int. Cl. A61K 31/343; A61P 25/00; A61P 25/24; A61P 3/04; A61P 3/10; A61P 25/08; A61P 25/06; A61P 25/28; C07D 307/81; A61P 25/16

(52) U.S. Cl. 514/469; 549/467

(57) ABSTRACT

This invention provides new 2,3-dihydro-benzofuran compounds, their use for the treatment or prevention of melatoninergic disorders and its compositions.

### FIELD OF THE ART

The present invention belongs to the field of compounds with activity on melatonin receptors, especially 2,3-dihydro-benzofurans, and more specifically acylated 5-alkoxy-2,3-dihydro-benzofuran-3-yl-alkyl amines.

### STATE OF THE ART

Insomnia is the most common sleep disorder and affects 20-40% of adults, with a frequency that increases with age. Insomnia has many causes. One of these is the interruption of the normal wakefulness-sleep cycle. This dyssynchrony may result in pathological changes. A potential therapeutic treatment that allows correcting said effect consists in re-synchronising the wakefulness-sleep cycle by modulating the melatoninergic system (Li-Qiang Sun, Bioorganic & Medicinal Chemistry Letters 2005, 15, 1345-49).

Melatonin is a hormone segregated by the pineal gland that is responsible for information on the light-dark cycles, for controlling the circadian rhythm in mammals and for modulating retinal physiology. Melatonin synthesis and its nightly secretion are controlled by the suprachiasmatic nucleus and synchronised by environmental light (Osamu Uchikawa et al., J. Med. Chem. 2002, 45, 4222-39; Pandi-Perumal et al., Nature Clinical Practice 2007, 3 (4), 221-228).

Melatonin secretion in humans occurs simultaneously to sleep at night, and the increase in melatonin levels is correlated with the increase in the desire to sleep during the evening.

In humans, the clinical applications of melatonin range from treatment of the delayed sleep phase syndrome to jet lag treatment, including treatment applied to night shift workers and as a hypnotic treatment.

Melatonin receptors have been classified as MT1, MT2 and MT3 based on pharmacological profiles. The MT1 receptor is located in the hypothalamus central nervous system, whereas the MT2 receptor is distributed throughout the central nervous system and the retina. The presence of MT1 and MT2 receptors has been described at the peripheral level. The MT1 and MT2 receptors are involved in a large amount of pathologies, the most representative of these being depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders. The MT3 receptor has been recently characterised as the homologue of the quinone reductase-2 (QR2) enzyme. MT1 and MT2 are G protein-coupled receptors (GPCR), the stimulation of which by an agonist leads to a reduction in adenylate cyclase activity and the resulting reduction in intracellular cAMP.

U.S. Pat. No. 4,600,723 and U.S. Pat. No. 4,665,086 advocate the use of melatonin to minimise alterations of the circadian rhythms that occur due to changes in work shifts from days to nights or from passing quickly through several time zones in an airplane (jet lag). Several families of compounds with melatoninergic activity had been described in patent documents EP 848699B1, U.S. Pat. No. 5,276,051, U.S. Pat. No. 5,308,866, U.S. Pat. No. 5,633,276, U.S. Pat. No. 5,708,005, U.S. Pat. No. 6,034,239 (ramelteon), U.S. Pat. No. 6,143,789, U.S. Pat. No. 6,310,074, U.S. Pat. No. 6,583,319, U.S. Pat. No. 6,737,431, U.S. Pat. No. 6,908,931, U.S. Pat. No. 7,235,550, WO 8901472 and WO 2005062992.

Patent application US 2005137247A1 describes a procedure for treating hypertension by means of compounds with agonist activity against the melatonin receptor belonging to formulas i-iv:

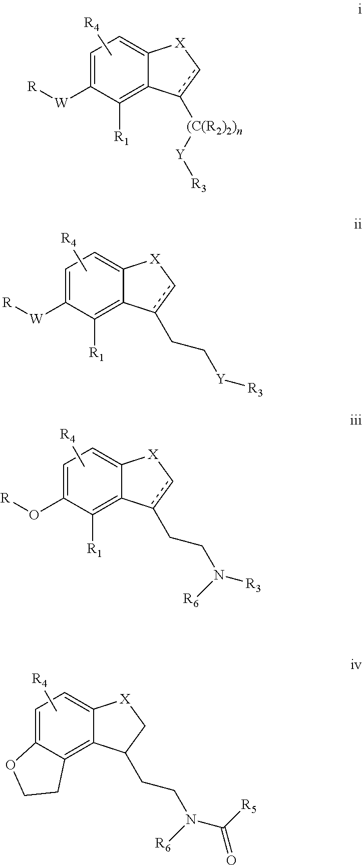

the different substituents and variables having the meanings described therein.

U.S. Pat. No. 6,147,110 describes benzofuran compounds belonging to formula:

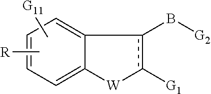

wherein the different substituents and variables have the meanings described therein, as well as their use in the treatment of diseases of the melatoninergic system.

Patent application WO 9608466 describes indane compounds as ligands to melatonin receptors belonging to formula:

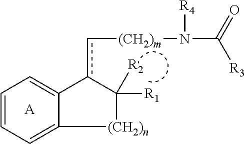

wherein substituents R₁, R₂, R₃ and R₄ and variables A, m and n have the meanings described therein.

Ramelteon, N-[2-[(8S)-1,6,7,8-tetrahydro-2H-indeno[5,4-b]furan-8-yl)ethyl]propionamide, is the first melatonin agonist introduced in therapy. It is indicated in insomnia and its mechanism of action is based on the agonism of the MT1 and MT2 receptors.

Ramelteon is a non-selective compound against MT1 and MT2, and selective against other receptors at the central and peripheral level. Its Ki is 0.014 nM for MT1 and 0.045 nM for MT2. It has resorption, but experiences an important first-pass metabolic effect. It is biotransformed into four metabolites, one of these being M-II, active and with an important distribution volume. Ramelteon clearance is 88%.

The research of new melatonin agonists that may be useful in the treatment of insomnia responds to a fundamental health need, and therefore justifies continued research for compounds with improved properties.

Therefore, the present invention is aimed at new acylated 5-alkoxy-2,3-dihydro-benzofuran-3-yl-alkyl amines active against melatonin receptors, particularly MT1 and MT2 receptors. As a result, the compounds of the present invention are useful in the treatment and prevention of all those diseases that are mediated by MT1 and MT2 receptors. Some non-limiting examples of melatoninergic disorders are depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

### DETAILED DESCRIPTION OF THE INVENTION

The present invention relates to 2,3-dihydro-benzofuran compounds of general formula I:

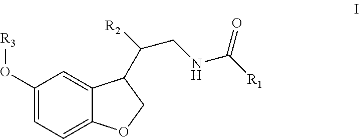

wherein:
R₁ is a radical chosen from the group consisting in a linear or branched (C₁-C₆) alkyl, NHR₄, (C₃-C₆) cycloalkyl and OR₅;
R₂ is hydrogen or a linear or branched (C₁-C₆) alkyl radical;
R₃ is a linear or branched (C₁-C₆) alkyl radical;
R₄ is a linear or branched (C₁-C₆) alkyl radical; and
R₅ is a linear or branched (C₁-C₆) alkyl radical; and pharmaceutically acceptable salts and hydrates thereof.

Pharmaceutically acceptable salts are those that may be administered to a patient, such as a mammal (e.g. salts with acceptable safety in mammals for a given dosing regimen). Such salts may be obtained from pharmaceutically acceptable inorganic and organic bases and from pharmaceutically acceptable inorganic and organic acids. The salts obtained from pharmaceutically acceptable inorganic bases include ammonium, calcium, copper, ferric and ferrous salts, lithium, magnesium, manganic and manganous salts, potassium, sodium, zinc salts and the like. Especially preferred are the ammonium, calcium, magnesium, potassium and sodium salts. The salts obtained from pharmaceutically acceptable organic bases include primary, secondary and tertiary amine salts, including substituted amines, cyclic amines, natural amines and the like, such as arginine, betaine, caffeine, choline, N,N′-2-dibenzylethylendiamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. The salts obtained from pharmaceutically acceptable acids include acetic, ascorbic, benzene sulphonic, benzoic, camphosulphonic, citric, ethanesulphonic, edisylic, fumaric, gentisic, gluconic, glucuronic, glutamic, hippuric, hydrobromic, hydrochloric, isethionic, lactic, lactobionic, maleic, malic, mandelic, methanesulphonic, mucic, naphthalenesulphonic, naphthalene-1,5-disulphonic, naphthalene-2,6-disulphonic, nicotinic, nitric, orotic, pamoic, pantothenic, phosphoric, succinic, sulphuric, tartaric, p-toluenesulphonic, xinafoic and the like. Particularly preferred are citric, hydrobromic, hydrochloric, isethionic, maleic, naphthalene-1,5-disulphonic, phosphoric, sulphuric and tartaric acids.

The specific compounds of Formula I are chosen from the group consisting of:
1) N-[2-(5-methoxy-2,3-dihydro-benzofuran-3-yl)-ethyl]-acetamide;
2) N-[2-(5-methoxy-2,3-dihydro-benzofuran-3-yl)-ethyl]-propionamide;
3) N-[2-(5-methoxy-2,3-dihydro-benzofuran-3-yl)-ethyl]-butyramide;
4) [2-(5-methoxy-2,3-dihydro-benzofuran-3-yl)-ethyl]-cyclopropanecarboxamide;
5) 1-ethyl-3-[2-(5-methoxy-2,3-dihydro-benzofuran-3-yl)-ethyl]-urea; and
6) Methyl [2-(5-methoxy-2,3-dihydro-benzofuran-3-yl)-ethyl]-carbamate.

Table 1 shows the meaning of the substituents for each compound:

TABLE 1

| Example | R₁ | R₂ | R₃ |
|---|---|---|---|
| 1 | Me | H | Me |
| 2 | Et | H | Me |
| 3 | Pr | H | Me |
| 4 | cPr | H | Me |
| 5 | NHEt | H | Me |
| 6 | OMe | H | Me |

Another aspect of the present invention is to provide the use of a specific compound from Table 1 to prepare a medicinal product for the treatment or prevention of melatoninergic disorders. Said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

Another aspect of the present invention is to provide pharmaceutical compositions comprising a specific compound from Table 1 and one or more pharmaceutically acceptable excipients.

Another aspect of the present invention is to provide the use of said pharmaceutical compositions in the preparation of a medicinal product for the treatment or prevention of melatoninergic disorders. Said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

How to obtain compounds of general formula I is described in the following diagrams, wherein the substituents R₁, R₂ and R₃ are as described above.

How to obtain the compounds of general formula I is described in the following Diagram 1, shown for R₂=H and R₃=Me.

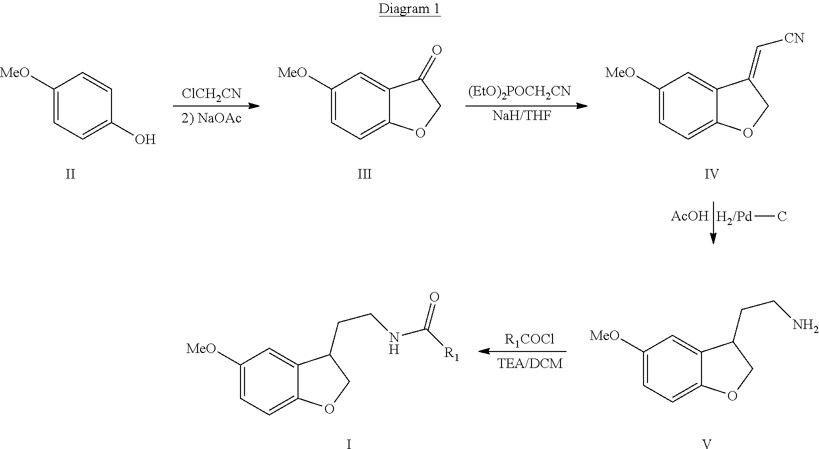

The first step consists in synthesising benzofuranone III, which is not commercially available. Benzofuranone III is thus obtained from 4-methoxyphenol II and by Friedel-Craft reaction with subsequent cyclization in a basic medium. The nitrile compound IV is then obtained by means of a Horner-Emmons reaction, using diethyl cyanomethyl sulphonate as a reagent. Said compound produces amine V by total hydrogenation of the cyano to amine and of the benzofuran to dihydrobenzofuran. Finally, the last step consists in a usual coupling between amines and acid chlorides to yield compounds I. Similarly, when the final products I are ureas or carbamates, the coupling reagents are the appropriate isocyanates or chloroformiates, respectively.

Pharmaceutical compositions comprising compounds of the present invention include those that are adequate for oral, rectal and parenteral administration (including the subcutaneous, intramuscular and intravenous routes), although the most suitable route will depend on the nature and seriousness of the pathology being treated. The preferred administration route for the compounds of the present invention is frequently the oral route.

The active ingredients can be mixed with one or more pharmaceutical excipients following conventional pharmaceutical techniques for formulation. Several excipients can be used according to the pharmaceutical form to be prepared. Liquid oral compositions (such as, for example, suspensions, solutions, emulsions, aerosols and mouthwashes) may use, for example, water, glycols, oils, alcohols, flavour enhancers, preservatives, colorants and the like. Solid oral compositions use, for example, starches, sugars (such as, for example, lactose, sucrose and sorbitol)celluloses (such as, for example, hydroxypropyl cellulose, carboxymethyl cellulose, ethyl cellulose and microcrystalline cellulose), talc, stearic acid, magnesium stearate, dicalcium phosphate, rubbers, copovidone, surfactants such as sorbitan monooleate and polyethyleneglycol, metallic oxides (such as, for example, titanium dioxide and ferric oxide) and other pharmaceutical diluents such as water. Homogeneous preformulations are thus formed containing the compounds of the present invention.

In the case of the preformulations the compositions are homogeneous, such that the active ingredient is dispersed uniformly in the composition, which can therefore be divided in equal unit doses such as tablets, coated tablets, powders and capsules.

Tablets and capsules are most advantageous oral forms due to their ease of administration. Tablets can be coated using aqueous or nonaqueous conventional techniques if so desired. A large variety of materials can be used to form the coating. Such materials include a large number of polymeric acids and their mixtures with other components such as, for example, shellac, cetyl alcohol and cellulose acetate.

Liquid forms in which the compounds of the present invention can be incorporated for oral or injectable administration include aqueous solutions, capsules filled with fluid or gel, syrups with flavour enhancers, aqueous suspensions in oil and emulsions flavoured with edible oils such as, for example, cottonseed oil, sesame oil, coconut oil or peanut oil, as well as mouthwashes and similar pharmaceutical carriers. Suitable dispersing or suspension agents for the preparation of aqueous suspensions include synthetic and natural gums such as tragacanth, Acacia, alginates, dextranes, sodium carboxymethylcellulose, methylcellulose, polyethyleneglycol, polyvinylpyrrodidone or gelatin.

A suitable dosage range to be used is a total daily dose from 0.1 to 500 mg approximately, more preferably from 1 mg to 100 mg, either in a single administration or in separate doses if necessary.

### EMBODIMENTS OF THE INVENTION

The present invention is additionally illustrated by means of the following examples, which do not intent to limit the scope thereof.

### Example of Pharmacological Assessment 1

### Determination of the Agonist Activity on MT1 Receptors

In order to screen compounds for the MT1 receptor a cell line is used that is characterised by stable overexpression of the recombinant human MT1 receptor in a cell line that in turn co-expresses mitochondrial apoaequorin and the Gα16 subunit.

The Gα16 subunit belongs to the G protein family, formed by GPCR, wherein the transduction of intracellular signals occurs via phospholipase (PLC). PLC activation produces an increase in inositol-triphosphate levels that leads to an increase in intracellular calcium. Gα16 overexpression thus allows an increase in intracellular calcium levels that is independent and compatible with the study receptor's own signal transduction pathway.

Apoaequorin is the inactive form of aequorin, a phosphoprotein that requires a hydrophobic prosthetic group, coelenterazine, to produce the active form. Following its binding to calcium, the aequorin oxidises coelenterazine to coelenteramide, a reaction that releases CO₂ and light.

The trial protocol for the screening of possible agonists consists in collecting the cells and keeping them in suspension overnight in the presence of coelenterazine in order to reconstitute aequorin. On the following day the cells are injected on a plate where the compounds to be screened are diluted, and the luminescence released is read immediately. When wishing to study the possible antagonism of the same compounds, the reference agonist compound is added in the same well after 15-30 min from the first injection and the luminescence released is assessed.

Agonist activity is calculated as percentage activity with respect to the reference agonist at the concentration corresponding to its EC100. Antagonist activity is expressed as percentage inhibition over the reference agonist activity at the concentration corresponding to its EC80.

### Example of Pharmacological Assessment 2

### Determination of Agonist Activity on MT2 Receptors

In order to study agonism against MT2 receptors we use a recombinant cell line that expresses these receptors and coexpresses mitochondrial apoaequorin and the Gα16 subunit, as in the model used for MT1 screening.

According to the methodologies described, the compounds of the present invention were verified to be powerful agonists of MT1 and MT2 receptors. Moreover, the compounds of the present invention advantageously provide relevant pharmacokinetic improvements. In this sense, the compounds of the present invention remarkably have better metabolic stability and better brain/plasma ratios than structurally similar compounds.

Therefore, studies of metabolic stability determined by the disappearance of the compounds to be tested by incubation in human microsomes for 120 min at 1 μM, and studies for the determination of levels in rat plasma (ng/mL) at 15 min after po administration of 1 mg/Kg of the compounds to be tested have shown that the compound from Example 1 has high metabolic stability (comprised between 71% and 100%) and plasma levels of 98 ng/mL and that the compound from Example 4 has an average metabolic stability (comprised between 31% and 70%), whereas comparatively (1S)-N-[2-(6-methoxy-indan-1-yl)-ethyl]-propionamide (WO 9608466 and O. Uchikawa et al., J. Med. Chem., 2002, 45, 4222-4239; compound 60) shows low metabolic stability (less than 30%), plasma levels of 10.1 ng/mL and a brain/plasma ratio close to zero. Consequently, the compounds of examples 1 and 4, despite certain structural similarities with the reference compound, show unexpectedly higher pharmacokinetic properties.

In short, the present invention provides new compounds that, despite having certain structural similarity with compounds of the state of the art, surprisingly show lower biotransformation, thus providing more sustained sleep.

### Reference Example 1

### General Procedure for Obtaining Benzofuranones III

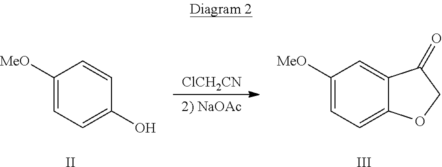

A solution of 48.3 mL of 1N BCl is cooled in an ice bath. A solution of 5 g (40.3 mmol) of 4-methoxyphenol in 100 mL of dichloroethane is added slowly to this solution at 0° C. for 1 h. A solution of 3.06 mL (48.3 mmol) of chloroacetonitrile in 10 mL of dichloroethane is added. 2.69 g (20.14 mmol) of AlCl3 are added in portions such that the temperature never exceeds 35° C. Stirring is continued at 0° C. for 2.5 h. Once the reaction has finished, the crude product is poured over a suspension of ice and 1N HCl. The phases are separated. The aqueous phase is extracted with 30 mL of dichloroethane. The organic extracts are gathered and dried over anhydrous magnesium sulphate.

The solvent is filtered and eliminated under low pressure. The residue thus obtained is redissolved in 100 mL of MeOH. 9.91 g (121 mmol) of sodium acetate are added at once and the mixture is boiled for 1 h and 30 min. It is allowed to cool and filtered. The filtrate is extracted with dichloromethane (DCM) and 1N NaOH. Dry, filter and evaporate the organic phase. 4.07 g (Yield=55%) of the benzofuranone III are obtained as a yellowish oil.

HPLC-MS: Purity 99%, M+1=165

### Reference Example 2

### General Procedure for Obtaining Compounds IV

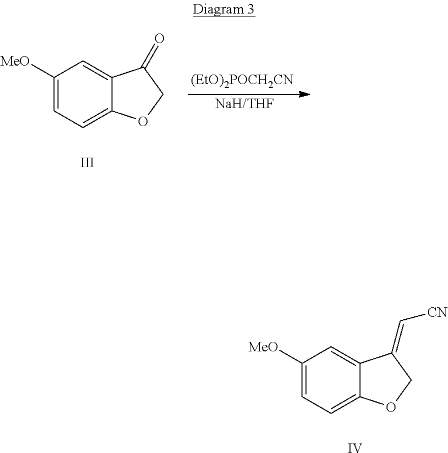

1.13 mL (24.79 mmol) of diethyl 1-cyanoethylphosphonate are dissolved in 100 mL of dry tetrahydrofurane (THF) and 0.99 g (24.79 mmol) of 60% NaH are slowly added. It is stirred for 1 h at room temperature. 4.07 g (24.79 mmol) of the methoxybenzofuranone III are added at once in 30 mL of dry THF. It is stirred at room temperature for 2 h more. 50 mL of water are added and it is stirred for 15 min at room temperature. The THF is evaporated at low pressure reduce and 100 mL of ethyl acetate are added to the resulting aqueous solution. The phases are separated and the organic phase is dried on anhydrous magnesium sulphate. The organic phase is evaporated and 2.44 g of oil are obtained. It is purified by column chromatography using ethyl acetate/hexane as an eluant mixture. 2.44 g of a yellow solid are thus obtained (Yield=53%).

HPLC-MS: Purity 100%, M+1=188

### Reference Example 3

### General Procedure for Obtaining Amines V

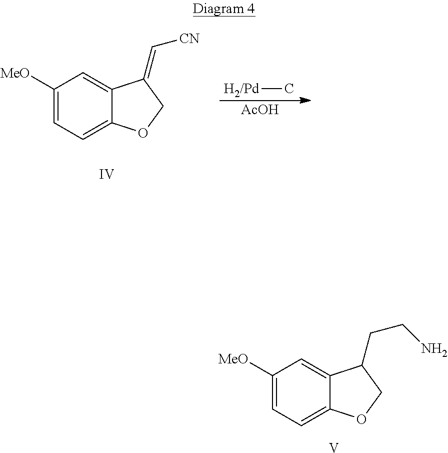

0.5 g (2.67 mmol) of the nitrile IV are dissolved in 50 mL of glacial acetic acid. 0.25 g of 10% Pd/C are added and it is introduced in a hydrogen atmosphere. It is stirred for 24 h. The catalyst is filtered and the solvent is evaporated from the filtrate under low pressure. 0.45 g of an oil are obtained that is suspended in 50 mL of 1M HCl and 50 mL of DCM are added. It is stirred for 20 min and the phases are separated. The aqueous phase is basified with 3N NaOH and extracted again with DCM. The organic phase is taken and dried over anhydrous magnesium sulphate. The solvent is filtered and eliminated under low pressure to produce 0.28 g of V (Yield=54%) as a yellowish oil.

HPLC-MS: Purity 100%, M+1=194

### Reference Example 4

### General Procedure for Obtaining Compounds I

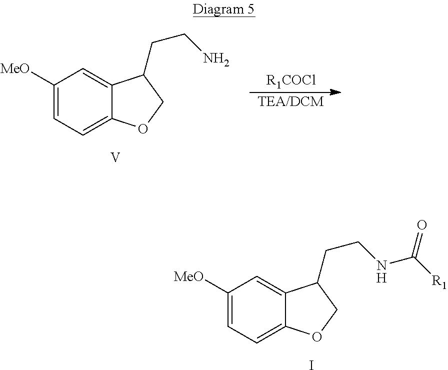

100 mg of amine V (0.209 mmol) are dissolved in 5 mL of anhydrous DCM. 0.053 mL of triethylamine (0.418 mmol) are slowly added and subsequently 0.23 mmol of the corresponding acid chloride are also slowly added. Stir at room temperature for 2 h and 30 min. 5 mL of 1N HCl are added and it is stirred for 10 min. Separate the organic phase and dry. It is evaporated to dryness. The residue thus obtained is purified by column chromatography using ethyl acetate/hexane as eluants. The final compounds I are thus obtained as a white solid.

Example when R₁=Et: 21 mg (Yield=41%) are obtained.

HPLC-MS: Purity 99%, M+1=250

The compounds thus obtained are detailed in the following Table 2.

TABLE 2

| Example | R₁ | R₂ | R₃ | LCMS Purity (%) | M + 1 |
|---|---|---|---|---|---|
| 1 | Me | H | Me | 99 | 236 |
| 2 | Et | H | Me | 99 | 250 |
| 3 | Pr | H | Me | 100 | 264 |
| 4 | cPr | H | Me | 100 | 262 |
| 5 | NHEt | H | Me | 99 | 265 |
| 6 | OMe | H | Me | 98 | 252 |

1. Compounds of 2,3-dihydro-benzofuran selected from the group consisting of:
1) N-[2-(5-methoxy-2,3-dihydro-benzofuran-3-yl)-ethyl]-acetamide;
2) N-[2-(5-methoxy-2,3-dihydro-benzofuran-3-yl)-ethyl]-propionamide;
3) N-[2-(5-methoxy-2,3-dihydro-benzofuran-3-yl)-ethyl]-butyramide;
4) [2-(5-methoxy-2,3-dihydro-benzofuran-3-yl)-ethyl]-cyclopropanecarboxamide;
5) 1-ethyl-3-[2-(5-methoxy-2,3-dihydro-benzofuran-3-yl)-ethyl]-urea; and
6) Methyl [2-(5-methoxy-2,3-dihydro-benzofuran-3-yl)-ethyl]-carbamate;
and pharmaceutically acceptable salts and hydrates thereof.

2. The use of a compound of claim 1 to prepare a medicinal product for the treatment or prevention of melatoninergic disorders.

3. The use of claim 2 wherein said melatoninergic disorders are selected from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

4. A pharmaceutical composition comprising a compound of claim 1 and one or more pharmaceutically acceptable excipients.

5. The use of the pharmaceutical composition of claim 4 to prepare a medicinal product for the treatment or prevention of melatoninergic disorders.

6. The use of claim 5 wherein the melatoninergic disorders are selected from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

7. A method of treating or preventing melatoninergic diseases that comprises administering an effective amount of one or more compounds of claim 1 to a patient.
